(19) United States
(12) Patent Application Publication
(10) Pub. No.: US 2010/0125217 A1
(43) Pub. Date: May 20, 2010

(54) Method and Apparatus for Presenting Heart Rate Variability by Sound and/or Light

(75) Inventors: Bo-Jau Kuo, Taipei (TW); Ching-Hsiu Yang, Taipei (TW)

Correspondence Address: HOGAN & HARTSON L.L.P., 1999 AVENUE OF THE STARS, SUITE 1400, LOS ANGELES, CA, 90067, US

(73) Assignee: National Yang-Ming University, Taipei (TW)

(21) Appl. No.: 12/409,730

(22) Filed: Mar. 24, 2009

(30) Foreign Application Priority Data
Nov. 17, 2008 (TW) 097144341

(51) Int. Cl. A61B 5/0472; A61B 5/04

(52) U.S. Cl. 600/515; 600/523

(57) ABSTRACT

An apparatus is disclosed for presenting heart rate variability, comprising a sensor for collecting electrocardiogram signals; an analog-to-digital converter for digitalizing electrocardiogram signals; an operating unit, converting said digitalized electrocardiogram signals to one or more heart rate variability parameters and further converting said heart-rate-variability parameters to one or more standard scores of heart rate variability by a standard mathematical formula; and an output unit, outputting the standard score of said heart-rate-variability by sound and/or light to aid determining the state of heart rate variability of a subject.

### BACKGROUND OF THE INVENTION

1. Field of the Invention

This invention relates to a method and a device that can present heart rate variability; more particularly, this invention relates to a method and a device that can present heart rate variability by sound and/or color.

2. Description of the Related Art

In the academicals and clinical field, there has been years of research on automatic nervous system. Currently, the most frequently used method is that analyzing the effect of the sympathetic nerve and parasympathetic nerve. Heart rate variability is mainly about analyzing the connection between Normal-to-Normal Intervals variability and physical reactions. The heart period herein refers to the intervals between every two heart beats. According to the publication on heart rate variability signal measure and analysis standard of European Society of Cardiology and North American Society of Pacing and Electrophysiology, the analysis of heart rate variability can be divided into time domain and frequency domain. Time domain analysis is to perform statistic and geometric calculations on Normal-to-Normal Intervals. Statistic calculations are, for example, mean, standard deviation (SD), coefficient of variation (CV), root mean square successive difference (RMSSD), Standard Deviation of Normal-to-Normal Intervals (SDNN), Standard Deviation of Standard Deviation of Normal-to-Normal Intervals (SDSD), etc during heart beat periods; moreover, geometric method can gain, for example, HRV triangular index, TINN, etc.

Frequency domain analysis is to convert the signal that changes with the time during the Normal-to-Normal intervals into the spectrum of Normal-to-Normal intervals and the strength of which is the square of the amplitude of the Sinusoidal wave of the frequency. After quantifying the relative strength, it becomes Power spectral density (PSD). With this method, any subtle wave motion can be made clear. Heart rate variability can be further divided into High-frequency (HF) and Low-frequency (LF). The total area under the power spectrum curve is the total power (TP) (the area in the high frequency area is the High-frequency power (HFP) and the area in the low frequency area is the Low-frequency power (LFP)). Likewise, in the standard of measure and analysis of the heart rate variability signal published by European Society of Cardiology and North American Society of Pacing and Electrophysiology in 1996, the definition of high frequency range is from 0.15 to 0.4 Hz, and the low frequency power might be related to adjustment of sympathetic nerve and parasympathetic nerve, and the contraction of renin vessel. Due to the complication of the mechanism of autonomic nervous system, there are various adjustment factors and these factors are difficult to examine. Therefore, the exact physical mechanism still needs further studies. At present, apart from high frequency and low frequency, researchers further define very low frequency (VLF) from the low frequency, the frequency range of the very low frequency is less than 0.04 Hz, and if it's a long term heart rate variability analysis (e.g. 12 or 24 hours), Ultra low-frequency (ULF) can further be found, the frequency range of which is less than 0.03 Hz, so as to examine autonomic nervous mechanism from a more detailed perspective.

Many physiologists have found that the high frequency of SDNN and heart rate, and the total power of heart rate variability present the Pneumogastric nerve (parasympathetic nerve) function of heart; while the ratio of the low frequency and the high frequency (LF/HF) can indicate the activity of the sympathetic nerve of heart. Previous research has shown that heart rate variability can indicate many physical functions. For example, the total power of the heart rate variability will reduce for the patients whose brain pressure has increased. The survey of the public health of Framingham in the US has shown that, if the low frequency of heart rate of elderly is a standard deviation lower, the chance of confronting death is 1.7 times more than an ordinary person. A series of software and hardware that can analyze the spectrums of various physical signals on-line in real time have been developed. For example, if the low frequency of heart rate and blood pressure is used as the indicator of the depth of anesthesia, in intensive care units, it can be found that when heart rate variability is lowered, the chance for patients to survive is lower and the low frequency of heart rate variability of brain dead patients disappears. Furthermore, if rejection occurs to heart transplantation patients, heart rate variability will also be changed.

In clinical medicine, there are many apparatuses and methods that diagnose automatic nerve function that have been developed, which comprise heart rate variation with deep breathing, valsalva response, sudomotor function, orthostatic blood pressure recordings, cold pressure test and biochemistry test. However, in the methods above, either the examinees have to go through a lot of pain during the examination, or the expensive apparatuses are needed. Moreover, some of the methods are not quite accurate or not convenient for operation, which also increases the difficulty in its applications.

From the current heart rate variability analysis, almost all machines present the range of heart rate variability by numbers. Although it is accurate, most people find it difficult to understand those numbers and it will take a lot training to interpret its meaning. Moreover, even for the personnel who are well trained, the numbers still need to be carefully read to interpret the numbers to be either too high or too low, and further infer possible physical meanings. Take the existing electrocardiographs for example; most of the electrocardiographs consist of homochromous graphs and lines without sound presenting the strength of the heart rate variability.

The U.S. Pat. No. 6,993,389 publishes an invention that can make sure if a patient is suitable for receiving the asynchronous treatment of the QRS complex wave width from the electrocardiographs from heart. This invention measures the start and end of the polarization of the ventricle to calculate the R-R intervals through implanting the electrodes in the patients' bodies. However, this invention might have the problem of noise when the electrodes are measuring, such that the medical personnel might misinterpret the results of the heart rate variability analysis.

Taiwan patents M327721, I225394, I245622, I289052, 200642660, 200726439, 200744531 and 200824650 all use HRV to diagnose different diseases or symptoms. The facilities and equipments used all use numbers to present the magnitude of heart rate variability. Although it is very accurate, most people can not make any usable interpretation to extract physical meanings without going through training.

This invention has corrected the flaws mentioned above to present the strength of heart rate variability with sound and/or color, such that formerly the heart rate variability analyses that are difficult to interpret are made easy to understand, which would further popularize the applications of heart rate variability analysis, so that it can be applied to the medication for patients or ordinary people, or even the house care.

### SUMMARY OF THE INVENTION

An object of this invention is to provide a device that can present heart rate variability.

Another object of this invention is to provide a device that can present the state of heart rate variability by a standard mathematic formula.

Yet another object of this invention is to provide a device that can present heart rate variability with sounds.

Yet another object of this invention is to provide a device that can present heart rate variability with colors.

An object of this invention is to provide a method of presenting heart rate variability state.

Another object of this invention is to provide a method of presenting heart rate variability state through a standard mathematic formula.

Another object of this invention is to provide a method of presenting heart rate variability with sounds.

Another object of this invention is to provide a method of presenting heart rate variability with colors.

The device that can present heart rate variability state of this invention includes:

a sensor, used for accessing electrocardiogram;

an analog-to-digital converter, used for digitalizing electrocardiogram;

an operation unit, used for converting the digitalized electrocardiogram into one or more heart rate variability parameters, and further convert the heart rate variability parameters into one or more heart rate variability standard score through a mathematic formula; and

an output unit, outputting the heart rate variability points with sounds and/or colors, so as to help interpret the state of the strength of examinees' heart rate variability.

Moreover, the method of presenting heart rate variability of this invention, includes the following steps:

accessing the electrocardiogram of the examinees;

converting the electrocardiogram into one or more interpretable heart rate variability parameters;

using the heart rate variability parameters gaining one or more heart rate variability standard score through a mathematic formula; and

converting the heart rate variability standard score into sounds and/or colors, so as to help interpret the heart rate variability state;

in which, the standard mathematical formula is: SC(x)=(x−meanx)/SDx, wherein meanxand SDx represent mean and standard deviation, respectively.

The sensor mentioned above can be any conventional sensors that can access electrocardiogram, such as cardiograph sensor.

The above operation units can be any conventional operation units, such as PC.

The above heart rate variability parameters, can be divided into high-frequency (HF), low-frequency (LF), total power (TP), the ratio of the low-frequency and High-frequency (LF/HF), the percentage that the low-frequency occupies the total power of the high and low frequency (LF %), mean of R-R intervals, standard deviation (SD), RMSSD, SDNN or SDSD.

The output unit mentioned above can be any conventional output unit, such as display, speakers and so on, along with the appropriate colors and/or sounds that indicate the heart rate variability after being interpreted, such that the user himself and operators (doctor or nurse) can know the state of the strength of the heart rate variability of the examinees.

The above mentioned standard mathematical formula SC(x)=(x−meanx)/SDx, wherein meanx and SDx are the mean and standard deviation of x, respectively, which is the same as the definitions in statistics. The meanx and SDx are used to calculate the heart rate variability standard score of each heart rate variability parameter.

Before calculating the standard score of each parameter, a database can be established to record the SDNN, TP, HF, LF/HF, LF % of different age, gender and patients with different diseases and get the mean and standard score. The mean of each parameter is used as the meanx of the standard score (SC function). The standard score of each parameter are surveyed according to different ages, genders and all kinds of diseases, and give it the strength of heart rate variability for future comparison.

When choosing colors for helping interpret the strength of heart rate variability of examinees, that is when users use this technology, the wave form of the electrocardiogram can be seen, and at the same time the strength of the heart rate variability of the examinees can be seen through the color of the wave form. As for the heart rate variability apparatus, it offers a very simple way of indication, such that users can know the state of heart rate variability of the examinees. The way of using color for indication can be any conventional way of using color to indicate different state of strength of heart rate variability, so that red, orange, yellow, green, blue, indigo, purple can be applied to the way of indication; or converting the heart rate variability electrographic signals into a series of visible spectrum color change, not simply two positional (e.g. change from red to yellow not directly but gradually).

When choosing sounds to help interpret the strength of the heart rate variability of the examinees, that is when users use this technology, the heart rate variability of the electrocardiogram can be heard, and at the same time the strength of the heart rate variability of the examinees can be known through the expression of sound. As for the heart rate variability apparatus, it offers a very simple way of indication, such that users can know the state of heart rate variability of the examinees by only listening to the sounds. The way of using sounds for indication can be any conventional way of using sounds to indicate different state of strength of heart rate variability, for example, by means of base frequency and frequency modulation. As for the electrocardiogram apparatus, the extra value can be added on the conventional electrocardiogram apparatus, but only in the case that the hardware is equipped with speaker; the electrocardiogram apparatus can have the function of showing heart rate variability. For example, when the heart rate variability is too low, the alert can be indicated by monotone frequency; and when the heart rate variability is too high, the alert can be indicated by any frequency modulation sound to present another alert.

Before sampling the electrocardiogram, the electrocardiogram still need to be screened to filter out the noise (please refer to FIG. 2 for the process of filtering), and then, sampling procedure and fixing value procedure are performed again to maintain the coherence of time. First of all, remove the straight line drift to prevent interference in low frequency area, and adopt Hamming operation to prevent leakage of each frequency in each spectrum. Then, Fast Fourier Transform is implemented to gain Heart rate Power Spectral Density (HPSD), and to complement the impact caused by sampling and Hamming operation, so as to reduce error. The power of the two of the heart rate spectrums in the heart rate spectrums are fixed through integration method, and the two heart rate spectrums include the low frequency power (LF) between 0.04-0.15 Hz and the high frequency power (HF) between 0.15-0.4 Hz. Meanwhile, total power (TP) of high and low frequencies, the ratio of the low frequency and high frequency (LF/HF) and the percentage that the low frequency occupies the total power (LF %) and other quantified parameters are gained. The SDNN, HF and TP relate to the activity of the parasympathetic nerve of heart. The LF/HF and LF % are the parameters that relate to the activity of the sympathetic nerve of heart; and the LF is the combined indication of sympathetic and parasympathetic nerves, that is the indication of autonomic nerve indication.

### BRIEF DESCRIPTION OF THE DRAWINGS

FIG. 1 schematically illustrates a preferred embodiment of this invention that the device can present the state of heart rate variability.

FIG. 2 schematically illustrates the QRS wave used when the device that can present heart rate variability extracts electrocardiogram.

FIG. 3 is the flow chart of this invention of the device presenting heart rate variability state with colors.

FIG. 4 is the flow chart of this invention of the device presenting heart rate variability state with sounds.

### DETAILED DESCRIPTION OF THE PREFERRED EMBODIMENTS

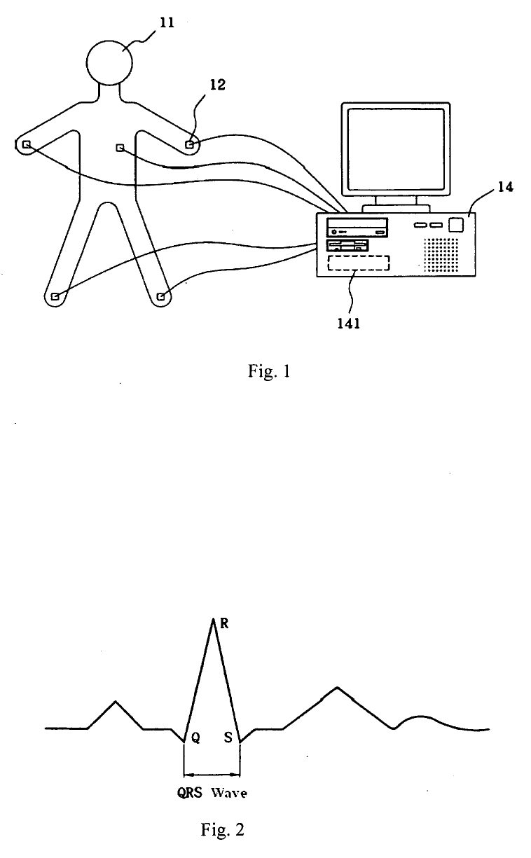

FIG. 1 schematically illustrates a preferred embodiment of the device that can present the state of heart rate variability. This invention uses electrode 12 as a heart beat sensor to collect a body's 11 electrocardiogram (ECG). After the ECG is enlarged 1000 times and 0.16-16 Hz band-pass is filtered out to input into a computer 14, and a analog-to-digital converter 141 contained in the computer 14 sample at the frequency of 256 times per second. The digitalized electrocardiogram can use a program on the computer to analyze the heart rate variability of a body 11 on-line immediately, and the results can be saved in the computer 14 to facilitate analysis and presence of sound and light. The electrode 12 can also be replaced by pressure sensor, microphone or photodiodes, as long as the device has the function of detecting ECG. This embodiment mainly makes use of a computer 14 containing an analog-to-digital converter 141 to store and analyze the ECG.

FIG. 2 schematically illustrates the QRS wave used by the device that can present heart rate variability state of this invention when accessing ECG. Generally speaking, the most bulging wave is called the QRS wave, wherein the Q spot is the first spot that bends upwards and the R spot is the top spot; finally, the S spot is at the bottom. During the procedure of QRS identification, the QRS wave of the ECG is first found out through peak detection procedure, and then the amplitude, duration and other parameters are measured from the QRS wave. Next, the mean and standard deviation of each QRS wave are gained to be the standard module. After that, each QRS is compared in accordance with the module. If the comparison result of a QRS wave falls out of three standard deviations of the standard module, the wave will be seen as noise or ectopic beat and be deleted. Afterwards, use the R spot of the accepted QRS wave as the time point of the heart beat, and the time difference between the present heart beat and the next heart beat is used as the R-R interval of the present heart beat. After that, the filter procedure of the R-R interval is implemented. First of all, the means and standard deviations of all heart beats are gained, and the filtering of the R-R intervals is then implemented. If a certain R-R interval falls out of the four standard deviations, it will be seen as an error or an unstable signal and removed.

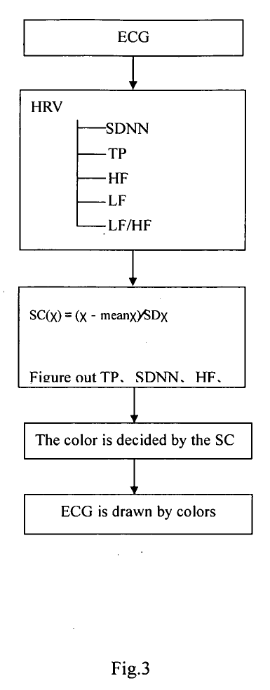

FIG. 3 is the flow chart of the device that can present heart rate variability state with colors of this invention. This invention first collects ECG, and then gains heart rate variability parameters through operation. The heart rate variability parameters include the SDNN gained by time division method, and the TP, HF, LF and LF/HF gained by frequency division method; and these parameters can all be the basis for showing colors. As for TP, a heart rate variability standard score is gained using the standard mathematic formula, SC(x)=(x−meanx)/SDx(The heart rate variability standard score of SDNN, HR, LF and LF/HF can also be gained through the aforementioned equation). Next, the colors used are decided by the heart rate variability standard score of TP. Finally, the ECG is drawed out with colors. After that, the strength of the heart rate variability can simply be told by the colors. Red, orange, yellow, green, blue, indigo, purple and other fake colors are used to present the strength of heart rate variability to present the heart rate variability in the color ECG, such that the ECG presented, at the same time, includes the results of heart rate variability analysis and interpretation.

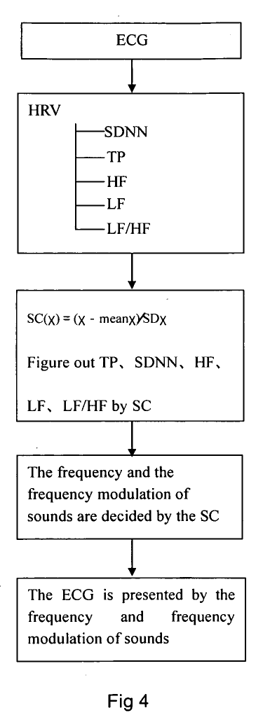

FIG. 4 is the flow chart of the device that can present heart rate variability state of this invention. This invention first collects ECG, and then gains heart rate variability parameters through operation. The heart rate variability parameters include the SDNN gained by time division method, and the TP, HF, LF and LF/HF gained by frequency division method; and these parameters can all be the basis for showing colors. As for TP, a heart rate variability standard score is gained using the standard mathematic formula, SC(x)=(x−meanx)/SDx(The heart rate variability standard score of SDNN, HR, LF and LF/HF can also be gained through the aforementioned equation). Next, the sounds used are decided by the heart rate variability standard score of TP. Finally, the ECG is presented with sounds. After that, the strength of the heart rate variability can simply be told by the sounds. Frequency and the frequency modulation of the sounds are used to present the strength of heart rate variability to present the heart rate variability in the ECG presented with sounds, such that the ECG presented, at the same time, includes the results of heart rate variability analysis and interpretation.

What is claimed is:

1. A device that can present heart rate variability, comprising:
a sensor for detecting electrocardiogram signals;
an analog to digital converter for digitalizing electrocardiogram signals;
an operation unit, converting said digitalized electrocardiogram signals to one or more heart rate variability parameters, and further converting said heart rate variability parameters to one or more standard scores of heart rate variability by a standard mathematical formula and out put unit, outputting the standard scores of said heart rate variability by sound and/or color for determining the state of heart rate variability of the examinee.

2. The device according to claim 1, wherein the sensor is the cardiograph sensor.

3. The device according to claim 1, wherein the parameter of said heart rate variability includes high-frequency(HF), low-frequency (LF), total power (TP), ratio of the low-frequency and high-frequency (LF/HF), the percentage that the low frequency occupies the total power of high frequency and low frequency (LF %), heart rate power density spectrum, mean of R-R intervals, standard deviation (SD), root mean square successive difference (RMSSD), Standard Deviation of Normal-to-Normal Intervals (SDNN), Standard Deviation of Standard Deviation of Normal-to-Normal Intervals (SDSD).

4. The device according to claim 1, wherein the color output corresponds to a function to be converted into a series of color change of visible light spectrum.

5. The device according to claim 1, wherein the sound output corresponds to a function to be converted into the sounds with different frequency and frequency modulation.

6. A method of presenting heart rate variability, comprising:
accessing the electrocardiogram signal from the examinee;
converting the electrocardiogram signal to one or more interpretable heart rate variability parameters;
gaining one or more standard score of heart rate variability by a standard mathematical formula according to the heart rate variability parameters; and
converting the heart rate variability standard score to sound and/or color by detecting the state of the heart rate variability of the examinee.

7. The method according to claim 6, wherein the standard mathematical formula is: SC(x)=(x−meanx)/SDx, wherein the meanxand the SDx represent the mean and the standard deviation of x respectively, andx may be any kind of heart rate variability parameters.

8. The method according to claim 6, wherein the method further includes a database, used for recording the SDNN, TP, HF, LF/HF, LF % and all kinds of heart rate variability parameter data of all ages, genders and/or all patients, and calculate the mean and the heart rate variability standard score thereof.

9. The method of detecting heart rate variability according to claim 8, wherein the database is used to compile statistics on the heart rate variability standard score from the perspectives of age, gender and/or all kinds of disease along with the strength of the heart rate variability for comparing.

10. The method of detecting heart rate variability according to claim 6, wherein the parameters of the heart rate variability are high-frequency(HF), low-frequency (LF), Total power (TP), ratio of the low-frequency and high-frequency (LF/HF), the percentage that the low frequency occupies the total power of high frequency and low frequency (LF %), heart rate power density spectrum, mean of R-R intervals, standard deviation (SD), RMSSD, SDNN or SDSD.

11. The method according to claim 10, wherein the heart rate power density is gained by converting the electrocardiogram signal using Fourier transfer.

12. The method according to claim 6, wherein the method of detecting heart variability further includes a step of QRS wave screening and/or R-R intervals.
